(19) United States
(12) Patent Application Publication
(10) Pub. No.: US 2003/0217744 A1
(43) Pub. Date: Nov. 27, 2003

(54) Bite block

(75) Inventors: Yukimi Sugai, Kanagawa-ken (JP); Hideaki Kito, Yamanashi-ken (JP)

Correspondence Address: GREENBLUM & BERNSTEIN, P.L.C., 1950 ROLAND CLARKE PLACE, RESTON, VA, 20191, US

(73) Assignee: Yukimi Sugai, Kanagawa (JP)

(21) Appl. No.: 10/442,970

(22) Filed: May 22, 2003

(30) Foreign Application Priority Data
May 24, 2002 (JP) 2002-151417

(51) Int. Cl. A61M 16/00

(52) U.S. Cl. 128/200.26

(57) ABSTRACT

A bite block, which not only prevents the blockage and the damage of an endo-tracheal tube, but also facilitates the observation of the oral cavity of patients, the bite block further having excellent effects of preventing ulceration in the upper and lower lips, the oral mucosa and the tongue.

The bite block comprises a pair of left and right bite portions, which are bitten by the left and right maxillar molar teeth, and a bridge connecting these portions, wherein the ceiling plane of each of the above pair of bite portions is in contact with a maxillar molar tooth on each of the left and right sides of a patient's mouth and the basal plane thereof is in contact with a submandibular molar tooth thereon, and the above bridge is molded in an arch shape, so as to fix the endo-tracheal tube.

### BACKGROUND OF THE INVENTION

1. Field of the Invention

The present invention relates to a bite block, which is inserted into the oral cavity to protect an endo-tracheal tube, when the endo-tracheal tube is used to assist the pulmonary respiration of patients.

2. Description of the Related Art

For an operation under general anesthesia or treatment of a gravely ill patient who can hardly maintain spontaneous respiration, an endo-tracheal tube is inserted into the trachea of the patient to ensure respiration using a respirator. In order to fix the inserted endo-tracheal tube, a bite block is used to prevent the deviation of the position of the tube, and to prevent the blockage or damage of the tube due to biting by the patient.

Conventional bite blocks as shown in FIGS. 7 and 8 have been widely used. These bite blocks have a configuration in which a flange 23 is formed close to either one of the ends of a rubber tubular main body 21. However, an oral tip 22 and the flange 23 of the tubular main body 21 press against the oral mucosa, the tongue, or the upper and lower lips, so that ulceration takes place. The development of such ulcer does not only bring about pain or become a soure of infection to a patient, but it also changes his or her complexion. This not only gives an unpleasant impression to the patient but also to his or her family, and can cause a mental burden for both the patient and his or her family.

Japanese Patent Laid-Open No. 8-47535 discloses a bite block comprising means for preventing the deviation of the position of an endo-tracheal tube. However, this bite block has a configuration in which a flange is positioned at both the front and back sides of the lip, so that the flange is always strongly in contact with the lip and the mucosa on the back side of the lip. Accordingly, there is a risk that ulceration might take place when the bite block is inserted for a long time.

Japanese Patent Laid-Open No. 2001-190675 discloses a bite block that is readily fixed. However, this bite block also comprises a ring C that is similar to the flange of the conventional bite block. The configuration of this bite block is such that the ring C is in contact with the lips and the oral tip of a barrel A is in contact with the tongue. Thus, the bite block has not solved the problem of ulceration in long-term use.

National Publication of International Patent Application No. 9-512457 discloses a bite block comprising a face plate, a head band and a tube-retaining block. However, this bite block has a configuration such that the face plate is likely to come into contact with and press against all or a part of the lips of a patient due to the fastening of the head band. Accordingly, this bite block has also not solved the problem of ulceration in long-term use.

Moreover, U.S. Pat. No. 5,386,821 discloses a bite block comprising left and right wedge-shaped portions, which are connected with each other by a U-shaped ridge. The publication states the effect of preventing the bite block from slipping into the oral cavity or pharynx. This is carried out by fixing the wedge-shaped portions by biting with the molar teeth, and positioning the U-shaped portion, which covers the maxillar tooth row other than the molar teeth, in front of the maxillar anterior teeth. However, since this bite block has a configuration such that the partial upper portion of the U-shaped portion comes into contact with the gingiva or upper lip mucosa, when the bite block is used for a long time, there is a risk that it might cause ulceration in the oral mucosa or the upper and lower lips.

The present invention was made to solve the above described problems. It is the object of the present invention not only to prevent the blockage or damage of an endo-tracheal tube, but also to prevent ulceration in the oral mucosa, the tongue and the lips due to long-term contact with an endo-tracheal tube and a bite block, and to achieve the treatment or improvement of the formed ulcer.

### SUMMARY OF THE INVENTION

The above described object can be achieved by the following features (1) to (8) of the present invention:

(1) A bite block used to fix an endo-tracheal tube into the oral cavity, the above bite block comprising a pair of bite portions and a bridge connecting these portions, wherein the ceiling plane of each of the above pair of bite portions is in contact with a maxillar molar tooth on each of the left and right sides of a patient's mouth and the basal plane thereof is in contact with a submandibular molar tooth thereon, and the above bridge is molded in an arch shape, so as to fix the endo-tracheal tube.

(2) The bite block according to (1) above, wherein the bite portion has a durometer hardness of A60 to D90.

(3) The bite block according to (1) above, wherein at least the ceiling plane of the above pair of bite portions of the bite block is subjected to non-slip working.

(4) The bite block according to (2) above, wherein at least the ceiling plane of the above pair of bite portions of the bite block is subjected to non-slip working.

(5) A bite block used to fix an endo-tracheal tube into the oral cavity, the above bite block comprising a pair of bite portions and a bridge connecting these portions, wherein the ceiling plane of each of the above pair of bite portions is in contact with a maxillar molar tooth on each of the left and right sides of a patient's mouth and the basal plane thereof is in contact with a submandibular molar tooth thereon, and the above bridge is formed in a shape which keeps a space for placing the endo-tracheal tube.

(6) The bite block according to (5) above, wherein the bite portion has a durometer hardness of A60 to D90.

(7) The bite block according to (5) above, wherein at least the ceiling plane of the above pair of bite portions of the bite block is subjected to non-slip working.

(8) The bite block according to (6) above, wherein at least the ceiling plane of the above pair of bite portions of the bite block is subjected to non-slip working.

### BRIEF DESCRIPTION OF THE DRAWINGS

FIG. 1 is a general front view showing an embodiment of the bite block of the present invention;

FIG. 2 is a view showing the bite block as shown in FIG. 1 from the top;

FIG. 3 is a sectional view, which is shown along the (III)-(III) line of FIG. 2;

FIG. 4 is a side view of the bite block as shown in FIG. 1;

FIG. 5 is a view showing a state such that the bite block as shown in FIG. 1 is inserted into the oral cavity;

FIG. 6 is a general front view showing another embodiment of the bite block of the present invention;

FIG. 7 is a view showing that the conventional bite block is inserted into the oral cavity; and

FIG. 8 is a sectional view showing that the conventional bite block is inserted into the oral cavity.

### DESCRIPTION OF THE PREFERRED EMBODIMENT(S)

The bite block of the present invention will be described below with reference to the figures attached hereto.

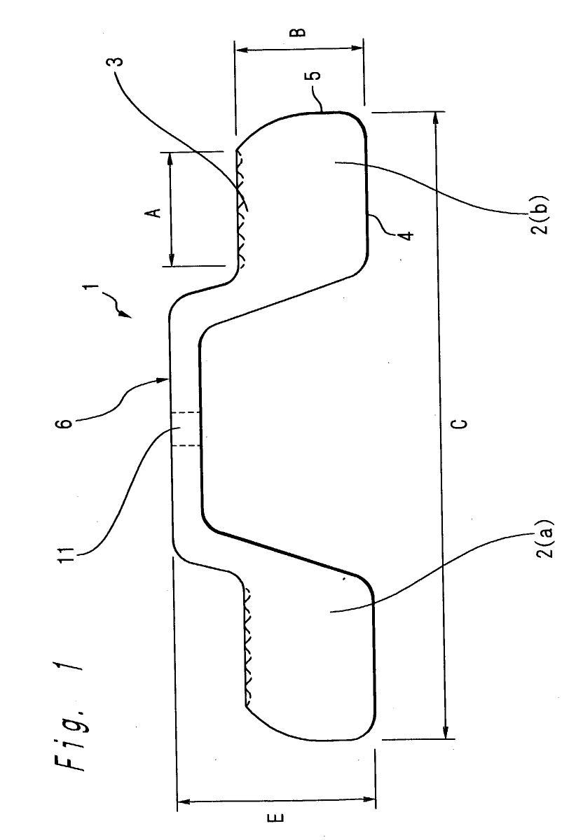

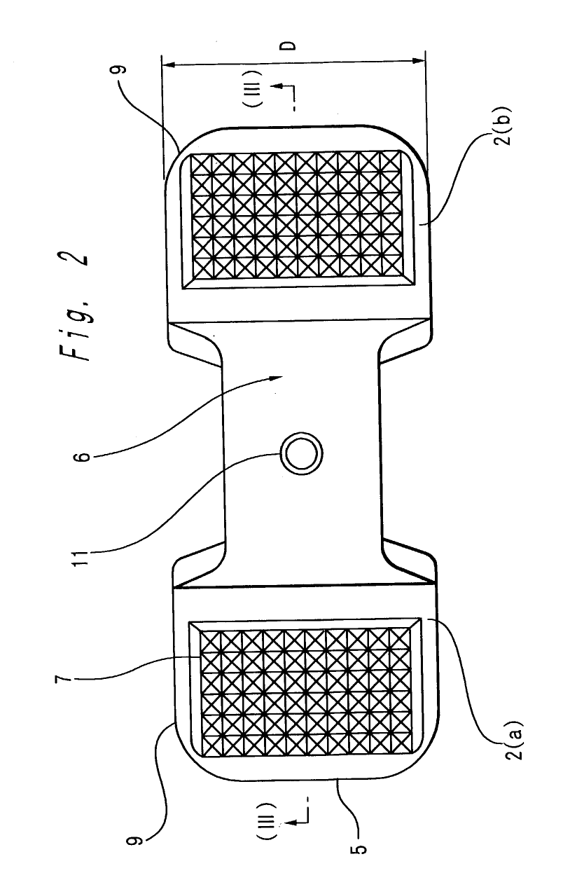

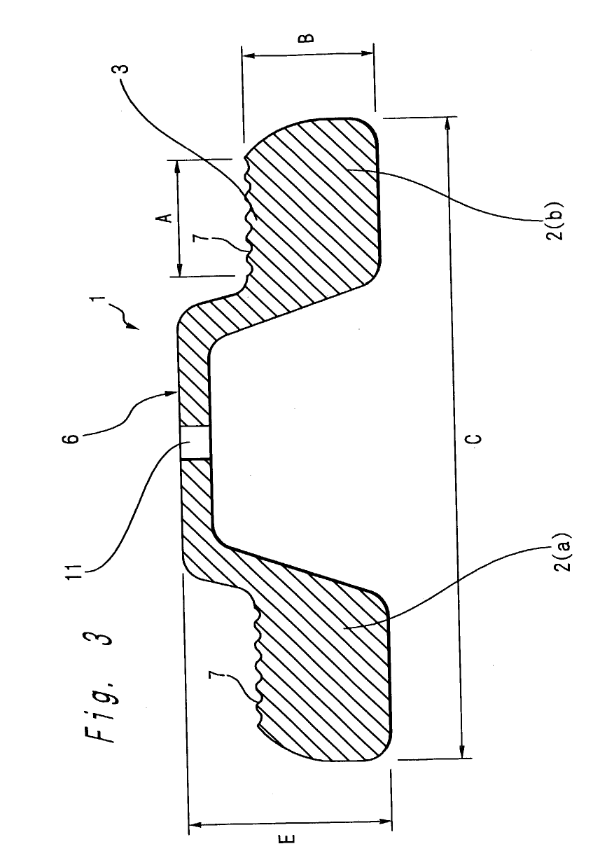

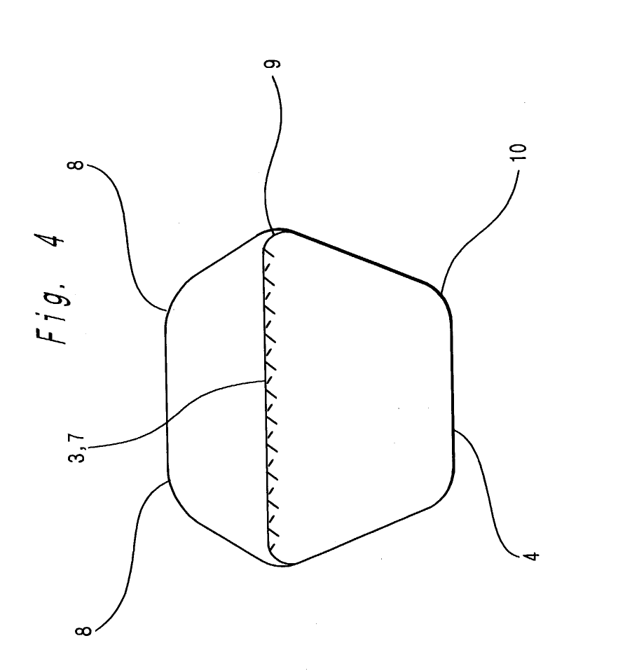

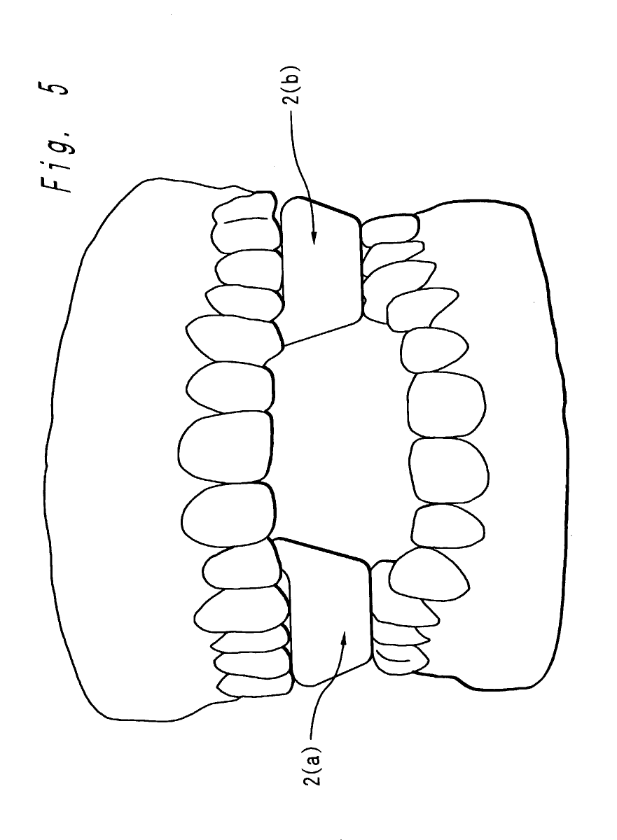

FIG. 1 is a general front view showing an embodiment of the bite block of the present invention. FIG. 2 is a view showing the bite block as shown in FIG. 1 from the top, FIG. 3 is a sectional view, which is shown along the (III)-(III) line of FIG. 2, and FIG. 4 is a side view of the bite block as shown in FIG. 1. Further, FIG. 5 is a view showing a state such that the bite block as shown in FIG. 1 is inserted into the oral cavity.

As shown in FIG. 1, a bite block 1 of the present invention comprises a pair of left and right bite portions 2(a) and 2(b), which are connected to each other by a bridge 6. The bite portion 2 (2(a) and 2(b)) has a thickness B sufficient to open the mouth of a patient, when it is inserted to the mouth. Specifically, the thickness B is preferably 5 to 20 mm. When the bite block is bitten by a patient, the width between the upper and lower anterior teeth is desirably 8 to 17 mm, and the heights of both the left and right sides are desirably the same to maintain stability when it is inserted to the mouth.

Moreover, the width A of each of the bite portions 2(a) and 2(b) is desirably the same as that of the molar tooth, or 120% or lower of the width of the molar tooth. It may be adequate, if the bite portions are molded so that a bite portion lateral face 5 and a peripheral portion 9 do not come into contact with the gingiva and that they are in contact with the buccal mucosa at the minimum. Specifically, the width A is preferably 5 to 15 mm. Further, when a patient is Japanese, the width A is more preferably 8 to 13 mm, which corresponds to the standard width of the molar tooth of a Japanese adult.

The length D of the bite portion 2 is preferably the same as the length of two molar tooth, or within the range of 15 mm to 30 mm, which corresponds to 120% or lower of the length of two molar tooth. Specifically, when a patient is Japanese, the length D is more preferably 19 to 25 mm, which corresponds to the standard length of two molar tooth of a Japanese adult.

The overall width C of the bite block is preferably within the range of 50 to 75 mm that is equivalent to the width of the dental arch. Moreover, when a patient is Japanese, approximately 64 mm that is the standard width of the dental arch of a Japanese adult is preferable.

By the way, dental arch index obtained by the following formula quantitatively expresses the form of the dental arch.

Dental arch index=Width of dental arch/Length of dental arch×100

Figures shown in the following table are data obtained by analyzing according to race, the above dental arch indexes obtained by the above formula.

Race Dental arch indexes of upper and lower jaws

Black upper jaw 105, lower jaw 122

Ceylonese upper jaw 108, lower jaw 117

Malayan upper jaw 117, lower jaw 126

Egyptian upper jaw 120, lower jaw 124

European upper jaw 125, lower jaw 148

Australian upper jaw 116, lower jaw 121

Chinese upper jaw 124, lower jaw 126

(Source: Ha no Kaibogaku [Dental Anatomy] (title), Tsunetaro Fujita (author), Kanehara & Co., Ltd. (publishing company))

Accordingly, it is preferable that the size of each part of the bite block of the present invention is set, as appropriate, depending on the race of a person who uses the present bite block. It is particularly preferable that the size is set, as appropriate, depending on the above described dental arch index for every different race.

Other documents “Koko no Kaibo [Anatomy of Oral Cavity] (title),pp. 11 to 19, Nanzando Co., Ltd. (publishing company) Kazuto Takahashi and Yoichiro Nosaka (authors and editing)” and “Koko Kaibogaku [Oral Cavity Anatomy] (title), pp. 132 to 193, Ishiyaku Pub., Inc. (publishing company), Sicher (author)” describe that “the male dental arch is slightly larger than the female dental arch, Australia's indigenous people and black people have a dental arch which is comparatively long from the front to the back of the mouth, white people have a comparatively wide dental arch, and that the yellow race has a middle type of dental arch between black and white people.”

Consequently, the size of the dental arch of Japanese who is the yellow race is the above described middle type, that is, the intermediate size of black and white people.

In the present embodiment, the bite block has a preferred embodiment such that the size of each part is set at the dental arch of the average Japanese, that is, the intermediate size of the dental arches of black and white people.

A ceiling plane 3 and a basal plane 4 of the bite portion are flat planes, so that they are fixed due to biting by the molar tooth. To prevent the deviation of the position of the bite block, non-slip working 7 maybe carried out. The surface of the non-slip working 7 desirably has a convexo-concave shape. However, the size or type of the convexo-concave shape is not limited, and it is adequate as long as the bite block is processed into a shape which hardly moves back and forth, and right and left. Moreover, the non-slip working may be carried out not only to the ceiling plane 3 but also to the basal plane 4.

The bridge 6 has an overall height E that is not in contact with the palatal mucosa. Moreover, the bridge is molded into an arch shape at an angle such that the bridge gradient portion is not in contact with the gingiva, so that a space for placing and fixing an endo-tracheal tube is ensured. The bridge 6 preferably has the overall height E such that the bridge 6 is not in contact with the upper jaw when it is attached to the molar teeth. Specifically, the overall height E of the bridge 6 is preferably 15 to 25 mm, and it is more preferably 17 to 20 mm.

Furthermore, it is also possible to establish on the bridge 6 a hole 11 for passing a string or thread, and thereby a string passing through the hole 11 is bound up to an endo-tracheal tube to fix the bridge 6, or a part of the string passing through the hole 11 is placed outside the oral cavity, so that it is used as a mark confirming that the bite block is used.

To reduce pressure against the oral mucosa or tongue to the minimum, all of the bite portion 2, the bite portion lateral face 5, and the peripheral portions 8, 9 and 10 of the bridge 6 are desirably formed with curved surfaces.

Examples of materials used for the bite block main body 1 include synthetic resins such as a plastic or elastomer, and such materials desirably have a strength such that the bite block main body is not easily impaired by the strength of the jaw.

Moreover, the ceiling plane 3 and the basal plane 4 of the bite portion are preferably made of a material softer than the molar tooth, so that the planes do not impair the teeth. Specifically, the bite portion 2 preferably has a durometer hardness of A60 to D90. The above hardness is determined according to JIS K 7215. Examples of preferred materials for the bite portion 2 include polypropylene, polyethylene, polyurethane, olefin elastomer, urethane elastomer, styrene elastomer, fiber reinforced plastics and others.

Furthermore, a part of the bite block main body 1 may be formed of metal. For example, the main body 1 other than the bite portions may be connected by a metal bridge. Still further, it may also be possible that the central base of the bite portion 2 is made of a material having a hardness such as metal and that the portion surrounding the central base is covered with a flexible material such as rubber. In this case, the bite block main body can directly be used to the gingiva.

The bite block of the present invention is basically inserted into a position such that the bite portions 2(a) and 2(b) are bitten by the upper and lower molar teeth. If a string or thread is passed through the threading hole 11, and the string or thread is bound up to an endo-tracheal tube for fixing or a part of the string or thread passing through the threading hole 11 is hung over the mouth, it can be confirmed that the bite block is used. The endo-tracheal tube is placed and fixed into a space formed by the bite portion 2 and the bride 6.

The endo-tracheal tube is fixed to some extent by being caught between the bridge 6 and the tongue. However, in order to ensure fixing, the tube is fastened at a certain site outside the oral cavity, using an adhesive tape or the like.

Further, if the string passing through the hole 11 is bound up to the endo-tracheal tube or the like for fixing, a case such that a patient might accidentally swallow the present bite block can be prevented.

Some specific examples are discussed below.

### EXAMPLE 1

A bite block having the length of an overall width C of 64 mm, a thickness B of the bite portion of 13.0 mm, a width A of the bite portion of 13.1 mm, a length D of the bite portion of 25.0 mm, an overall height E of 20.0 mm, made from polyethylene (a durometer hardness being D65), was inserted and fixed to the molar teeth in the oral cavity of each of 32 male and female adult patients. Thereafter, a respiratory tube was inserted into the trachea of each of the above patients, and the state in their oral cavity was observed on every 8 hours until the tube was removed. As a result, no ulceration was observed in the oral mucosa and the surface of the tongue of the 26 patients. Moreover, there were no patients who bit the endo-tracheal tube and blocked it. The period when the bite block was inserted was from a same-day removal to 37 days at longest.

### EXAMPLE 2

To 21 male and female patients who had an appearance of such as rubor, bleeding and ulceration by the insertion of the conventional bite block, the same bite block as in Example 1 was applied instead of the conventional one. Six hours to 8 days later, the symptom of the 12 patients was improved.

### COMPARATIVE EXAMPLE

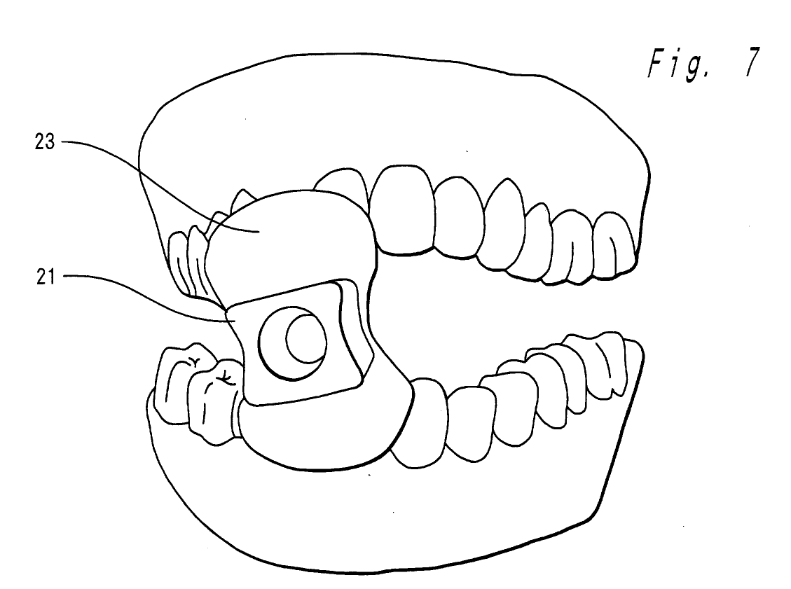

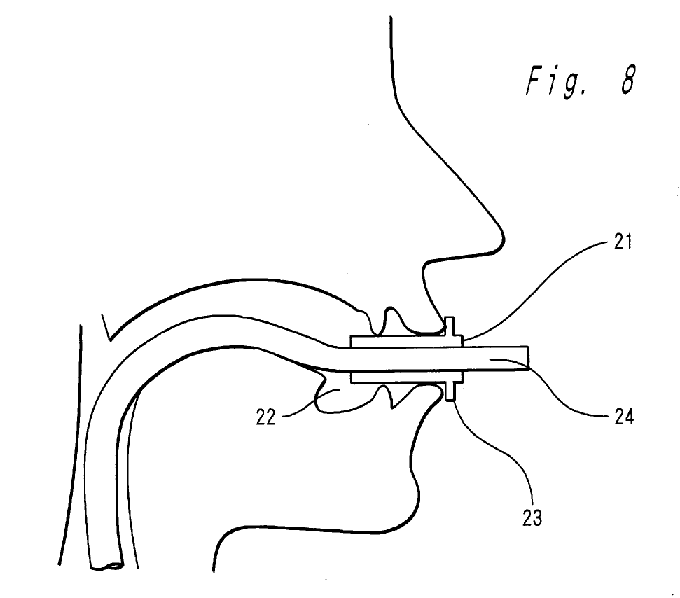

An endo-tracheal tube was inserted into the trachea of each of 27 male and female adult patients, and thereafter, the conventional bite block as shown in FIGS. 7 and 8 was inserted from the rear end of the above tube and fixed to the anterior teeth in the oral cavity. During the period from 4 hours or longer to shorter than 27 hours, symptoms such as rubor, bleeding or ulceration were appeared on the lips or tongue of the 21 patients.

The bite block of the present invention was explained as above based on the illustrated embodiments. However, the present invention is not limited thereto.

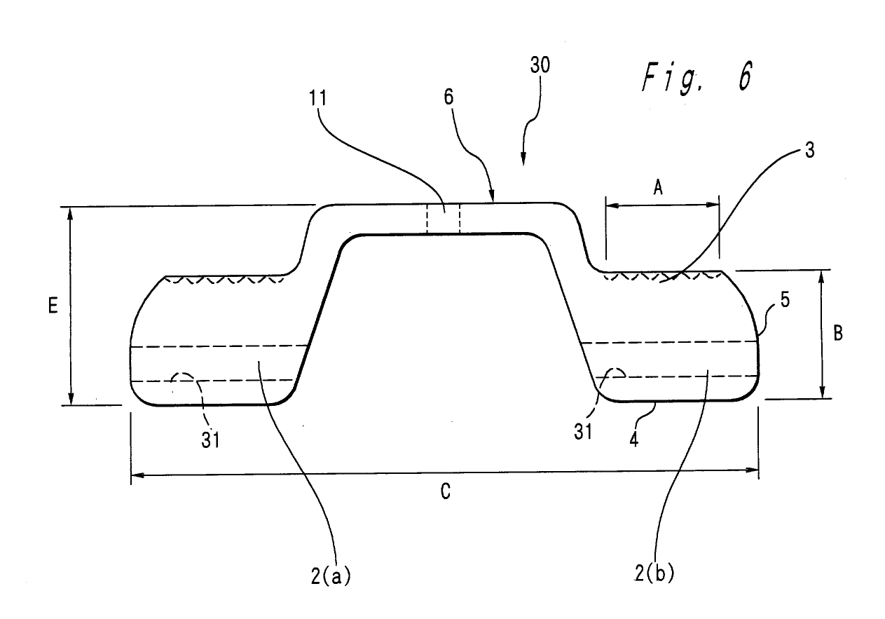

For example, FIG. 6 is a general front view showing another preferred embodiment of the bite block of the present invention.

In the figure, a bite block 30 has a configuration such that an airway 31 is added to the bite block 1 with the above configuration.

The airway 31 is formed so that it is passed through each of the pair of left and right bite portions 2(a) and 2(b) in the longitudinal direction of the bite block 30.

This bite block 30 can ensure the maintenance of a patient's airway for respiring by the airway 31, even if the patient swallows the bite block 30 and the block is lodge in the throat.

Moreover, in the above embodiment, the bridge 6 is molded into an arch shape as a preferred embodiment. However, the bridge 6 can be molded into any shape, as long as a space for placing an endo-tracheal tube is ensured. For example, the bridge 6 may have a shape such that it stretches over the pair of let and right bite portions 2(a) and 2(b), having a penetrating hole capable of passing the endo-tracheal tube there through.

As stated above, the bite block of the present invention does not only provide the effect of preventing the damage of an endo-tracheal tube, but also facilitates the observation of the oral cavity because a patient's mouth is opened from the anterior teeth part. When the bite block is used over a longtime, it can provide the effect of preventing ulceration in the oral mucosa, the tongue and the lips of a patient, or the effect of treating or improving the formed ulcer. Thus, the present bite block alleviates the mental burdens of a patient's family as well as the physical and mental burdens of the patient. Moreover, the present bite block reduces an opportunity of the administration of antibiotics for the prevention of infection, thereby contributing medical economic efficiency.

Having described specific preferred embodiments of the invention with reference to the accompanying drawings, it will be appreciated that the present invention is not limited to those precise embodiments, and that various changes and modifications can be effected therein by one of ordinary skill in the art without departing from the scope of the invention as defined by the appended claims.

What is claimed is:

1. A bite block used to fix an endo-tracheal tube into the oral cavity,
said bite block comprising a pair of bite portions and a bridge connecting these portions, wherein the ceiling plane of each of said pair of bite portions is in contact with a maxillar molar tooth on each of the left and right sides of a patient's mouth, and the basal plane thereof is in contact with a submandibular molar tooth thereon,
and said bridge is molded in an arch shape, so as to fix the endo-tracheal tube.

2. The bite block according to claim 1, wherein the bite portion has a durometer hardness of A60 to D90.

3. The bite block according to claim 1, wherein at least the ceiling plane of said pair of bite portions of said bite block is subjected to non-slip working.

4. The bite block according to claim 2, wherein at least the ceiling plane of said pair of bite portions of said bite block is subjected to non-slip working.

5. A bite block used to fix an endo-tracheal tube into the oral cavity,
said bite block comprising a pair of bite portions and a bridge connecting these portions, wherein the ceiling plane of each of said pair of bite portions is in contact with a maxillar molar tooth on each of the left and right sides of a patient's mouth, and the basal plane thereof is in contact with a submandibular molar tooth thereon,
and said bridge is formed in a shape which keeps a space for placing the endo-tracheal tube.

6. The bite block according to claim 5, wherein the bite portion has a durometer hardness of A60 to D90.

7. The bite block according to claim 5, wherein at least the ceiling plane of said pair of bite portions of said bite block is subjected to non-slip working.

8. The bite block according to claim 6, wherein at least the ceiling plane of said pair of bite portions of said bite block is subjected to non-slip working.
